(19) United States
(12) Patent Application Publication
(10) Pub. No.: US 2024/0368621 A1
(43) Pub. Date: Nov. 7, 2024

(54) ALBUGO CANDIDA RESISTANCE GENE

(71) Applicant: RIJK ZWAAN ZAADTEELT EN ZAADHANDEL B.V., De Lier (NL)

(72) Inventors: Catharina Fenny Jeanne DIJKHUIZEN, De Lier (NL); Marcus Jan Pieter DE JONG, De Lier (NL); Pieternella Jozina DE PREE, De Lier (NL)

(21) Appl. No.: 18/673,456

(22) Filed: May 24, 2024

Related U.S. Application Data
(63) Continuation-in-part of application No. PCT/EP2022/083272, filed on Nov. 25, 2022.

(30) Foreign Application Priority Data
Nov. 25, 2021 (WO) PCT/EP2021/083028

(51) Int. Cl. C12N 15/82; A01G 24/00; A01H 6/20; C12Q 1/6895

(52) U.S. Cl. CPC C12N 15/8282; A01G 24/00; A01H 6/206; C12Q 1/6895; C12Q 2600/13; C12Q 2600/156

(57) ABSTRACT

The present invention relates to a gene encoding a protein which confers resistance to Albugo candida when expressed in a Raphanus sativus plant, wherein said gene comprises:
a) a nucleotide sequence encoding a protein having an amino acid sequence, which has in order of increased preference at least 70%, 75%, 80%, 83%, 85%, 86%, 87%, 88%, 89%, 90%, 91%, 92%, 93%, 94%, 95%, 96%, 97%, 98%, 99%, 100% sequence similarity to SEQ ID No. 3; or
b) a nucleotide sequence comprising a coding sequence which has in order of increased preference at least 70%, 75%, 80%, 83%, 85%, 86%, 87%, 88%, 89%, 90%, 91%, 92%, 93%, 94%, 95%, 96%, 97%, 98%, 99%, 100% sequence identity to SEQ ID No. 2; or
c) a nucleotide sequence encoding at least the amino acid positions 15 to 192 of SEQ ID No. 3, the amino acid positions 209 to 432 of SEQ ID No. 3, and the amino acid positions 540 to 1023 of SEQ ID No. 3; or
d) a nucleotide sequence encoding an LRR domain comprising the amino acid positions 540 to 1023 of SEQ ID No. 3 or encoding an LRR domain which has in order of increased preference at least 95%, 96%, 97%, 98%, 99% sequence similarity to the amino acid sequence of the amino acid positions 540 to 1023 of SEQ ID No. 3.

### RELATED APPLICATIONS AND INCORPORATION BY REFERENCE

This application is a continuation-in-part of copending International Patent Application No. PCT/EP2022/083272, filed Nov. 25, 2022, which claims priority to International Patent Application No. PCT/EP2021/083028, filed Nov. 25, 2021, the contents of which are incorporated by reference herein in their entirety.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The instant application contains a Sequence Listing which has been submitted electronically and is hereby incorporated by reference in its entirety. Said XML copy, was created on May 6, 2024, is named Y7954-00593SL.xml, and is 17,739 bytes in size.

### FIELD OF THE INVENTION

The present invention relates to a gene conferring resistance against Albugo candida, and plants comprising said gene. The invention further relates to progeny, seed, and plant parts such as tubers and leaves of said resistant plant; and the invention relates to propagation material suitable for producing said plant. The invention also relates to markers for identifying the gene and resistant plant, the use of said markers, and to methods for selecting and producing the resistant plant.

### BACKGROUND OF THE INVENTION

White rust also known as white blister is a plant disease that is caused by a pathogen named Albugo candida. Like many other Oomycete species, Albugo candida is an obligate plant pathogen. It is capable of infecting all species within the Brassicaceae plant family, which comprises many economically important crop species such as e.g. Brassica napus, Brassica oleraceae, Diplotaxis tenuifolium, and Sinapis alba. Albugo candida is a widespread pathogen and occurs in almost all regions of the world where crops of the Brassicaceae family are grown. Especially in temperate areas with heavy dew Albugo candida can cause significant damage.

Infection by Albugo candida can occur both locally and systemically. Infection is commonly recognizable by the appearance of white colored blisters (pustules) on the leaves, stems or flowers. Systemic infection of a plant will lead to distorted growth and sterile inflorescences. The pathogen can survive in the soil, plant debris and in infected seeds. Sporangia that are formed in the pustules can spread via water, wind or insects and so infect other plants. Although there are fungicides available that can help to control Albugo candida, several strains have emerged that are resistant against one or more types of fungicide. Furthermore, increasing environmental regulations and the increased demand for organically grown crops demand a different approach in controlling this disease.

Radish (Raphanus sativus L.) is an edible plant species belonging to the Brassicaceae plant family. Although the entire plant is edible, they are mostly cultivated for their swollen hypocotyl sometimes including a swollen taproot. The skin of the swollen hypocotyl can have many different colors such as white, pink, red, purple, yellow, green and even black. The swollen hypocotyl, in some cases including the tap root, can have a globular, tapering or cylindrical shape and range in size.

Also, in radish cultivation—radish is typically grown in temperate areas—Albugo candida can cause severe damage. Although there are resistant varieties in radish, these varieties are not resistant to all strains of Albugo candida capable of infecting radish. For example, plants of patented inbred line NIZ-AC2 did not show resistance to all strains of Albugo candida. The resistance conferring QTL of NIZ-AC2 was mapped to chromosome 9 according to the publicly available genome assembly Rs1.0 of Raphanus sativus cv. WK10039 (www.ncbi.nlm.nih.gov/Traces/wgs/?val=JRUI02&display=contigs&page=1; as e.g. used and described in Mun J H, et al. (2015). Construction of a reference genetic map of Raphanus sativus based on genotyping by whole-genome resequencing. Theor. Appl. Genet. 128: 259-272.

Therefore, there is a need for improved genetic resistance to Albugo candida in Raphanus sativus.

Citation or identification of any document in this application is not an admission that such document is available as prior art to the present invention.

### SUMMARY OF THE INVENTION

In the research that led to the present invention a radish plant (Raphanus sativus L.) comprising a new gene providing resistance to Albugo candida was identified. It was surprisingly found that the resistance resulted from a TIR-NBS-LRR gene.

In the publicly available genome assembly Rs1.0 of Raphanus sativus cv. WK10039 (www.ncbi.nlm.nih.gov/Traces/wgs/?val=JRUI02&display=contigs&page=1; as e.g. used and described in Mun J H, et al. (2015). Construction of a reference genetic map of Raphanus sativus based on genotyping by whole-genome resequencing. Theor. Appl. Genet. 128: 259-272, the gene of the invention is located on chromosome 6 between 21,713,124 and 21,740,952 bp.

In the course of this research, it was further determined that in plants of line NIZ-AC2 the gene of the invention is not present.

The NBS-LRR family is the largest class of known R-proteins in the plant kingdom, providing resistance to a wide array of pathogens and abiotic stress factors. NBS-LRR proteins can be divided into TIR and Non-TIR-NBS-LRR proteins. TIR-NBS-LRR proteins are recognizable by three main domains. First, the TIR domain, which comprises a Toll/Interleukin-1 receptor structure. Secondly, an NBS (nucleotide binding site) domain, also known as NB-ARC domain, can be recognized. This is the main structural domain of the NBS-LRR genes. The third domain is the LRR (Leucine Rich Repeat) domain, which shows more variation than the previous two domains. The LRR domain is involved in the protein-protein interaction with the pathogen and assumed to play a key role in providing resistance.

Thus, the present invention provides a gene, herein referred to as the ‘gene of the invention’, encoding a protein which confers resistance to Albugo candida when expressed in a Raphanus sativus plant, wherein said gene comprises:
a) a nucleotide sequence encoding a protein having an amino acid sequence, which has in order of increased preference at least 70%, 75%, 80%, 83%, 85%, 86%, 87%, 88%, 89%, 90%, 91%, 92%, 93%, 94%, 95%, 96%, 97%, 98%, 99%, 100% sequence similarity to SEQ ID No. 3; or
b) a nucleotide sequence comprising a coding sequence which has in order of increased preference at least 70%, 75%, 80%, 83%, 85%, 86%, 87%, 88%, 89%, 90%, 91%, 92%, 93%, 94%, 95%, 96%, 97%, 98%, 99%, 100% sequence identity to SEQ ID No. 2; or
c) a nucleotide sequence encoding at least the amino acid positions 15 to 192 of SEQ ID No. 3, the amino acid positions 209 to 432 of SEQ ID No. 3, and the amino acid positions 540 to 1023 of SEQ ID No. 3; or
d) a nucleotide sequence encoding an LRR domain comprising the amino acid positions 540 to 1023 of SEQ ID No. 3 or encoding an LRR domain which has in order of increased preference at least 95%, 96%, 97%, 98%, 99%, 100% sequence similarity to the amino acid sequence of the amino acid positions 540 to 1023 of SEQ ID No. 3.

The gene of the invention is a nucleic acid, in particular a nucleic acid molecule, more in particular an isolated nucleic acid molecule.

The gene of the invention comprises a nucleotide sequence encoding a protein having an amino acid sequence, which has in order of increased preference at least 70%, 75%, 80%, 83%, 85%, 86%, 87%, 88%, 89%, 90%, 91%, 92%, 93%, 94%, 95%, 96%, 97%, 98%, 99% sequence similarity to an amino acid sequence according to SEQ ID No. 3.

Preferably, the gene of the invention comprises a nucleotide sequence encoding a protein having an amino acid sequence, which has at least 95% sequence similarity to an amino acid sequence according to SEQ ID No. 3. This nucleotide sequence preferably comprises at least a nucleotide sequence encoding an LRR domain comprising the amino acid positions 540 to 1023 of SEQ ID No. 3.

In a preferred embodiment, the gene of the invention comprises a nucleotide sequence encoding a protein having an amino acid sequence according to SEQ ID No. 3.

The gene of the invention comprises a coding sequence which has in order of increased preference at least 70%, 75%, 80%, 83%, 85%, 86%, 87%, 88%, 89%, 90%, 91%, 92%, 93%, 94%, 95%, 96%, 97%, 98%, 99% sequence identity to the coding sequence according to SEQ ID No. 2.

Preferably, the gene of the invention comprises a coding sequence which has at least 85% sequence identity to the coding sequence according to SEQ ID No. 2.

More preferably, the gene of the invention comprises a coding sequence which has at least 95% sequence identity to the coding sequence according to SEQ ID No. 2. This coding sequence preferably comprises at least a nucleotide sequence encoding an LRR domain comprising the amino acid positions 540 to 1023 of SEQ ID No. 3.

In a preferred embodiment, the gene of the invention comprises a coding sequence according to SEQ ID No. 2.

Furthermore, the gene of the invention comprises a nucleotide sequence comprising a genomic sequence according to SEQ ID No. 1, or a nucleotide sequence which has in order of increased preference at least 70%, 75%, 80%, 83%, 85%, 86%, 87%, 88%, 89%, 90%, 91%, 92%, 93%, 94%, 95%, 96%, 97%, 98%, 99% sequence identity to SEQ ID No. 1.

Preferably, the gene of the invention comprises a coding sequence which has at least 85% sequence identity to the genomic sequence according to SEQ ID No. 1.

More preferably, the gene of the invention comprises a genomic sequence which has at least 95% sequence identity to the genomic sequence according to SEQ ID No. 1. This genomic sequence preferably comprises at least a nucleotide sequence encoding an LRR domain comprising the amino acid positions 540 to 1023 of SEQ ID No. 3.

In a preferred embodiment, the gene of the invention comprises a genomic sequence according to SEQ ID No. 1.

The three main domains that can be recognized in the protein encoded by the gene of the invention are: 1) The TIR domain as defined herein by the amino acids on position 15 to 192 in SEQ ID No. 3. 2) The NBS domain as defined herein by the amino acids on position 209 to 432 in SEQ ID No. 3. 3) The LRR domain as defined herein by the amino acids on position 540 to 1023 in SEQ ID No. 3.

In one embodiment, the gene of the invention comprises a nucleotide sequence encoding a protein comprising the LRR domain on position positions 540 to 1023 of SEQ ID No. 3.

In a further embodiment, the gene of the invention comprises a nucleotide sequence encoding a protein comprising at least the amino acid positions 15 to 192 of SEQ ID No. 3, the amino acid positions 209 to 432 of SEQ ID No. 3, and the amino acid positions 540 to 1023 of SEQ ID No. 3.

The gene of the invention may further comprise a nucleotide sequence encoding a protein derived from the protein according to SEQ ID No. 3 by substitution, deletion and/or addition of one or more amino acids.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112(a)) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### DEPOSITS

The Deposits with NCIMB Ltd, Ferguson Building, Craibstone Estate, Bucksburn, Aberdeen AB21 9YA, UK, on Feb. 19, 2021, under deposit accession number 43737 were made and accepted pursuant to the terms of the Budapest Treaty. The seed of deposit NCIMB 43737 comprises the gene of the invention homozygously. Upon issuance of a patent, all restrictions upon the deposit will be removed, and the deposit is intended to meet the requirements of 37 CFR §§ 1.801-1.809. The deposit will be irrevocably and without restriction or condition released to the public upon the issuance of a patent and for the enforceable life of the patent. The deposit will be maintained in the depository for a period of 30 years, or 5 years after the last request, or for the effective life of the patent, whichever is longer, and will be replaced if necessary during that period.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description, given by way of example, but not intended to limit the invention solely to the specific embodiments described, may best be understood in conjunction with the accompanying drawings.

FIG. 1A-1F—Raphanus sativus plants in a young plant test classified according to the scale as presented in Table 2. The pictures show plants for each classification according to the scale of Table 2. FIG. 1A-1B: plants scoring resistant, i.e. showing no symptoms; FIG. 1C-1D plants scoring intermediate resistant, showing only minor symptoms with a single or just a couple of pustules; FIG. 1E-1F: plants scoring susceptible, lobs and leaf covered with white pustules of Albugo candida.

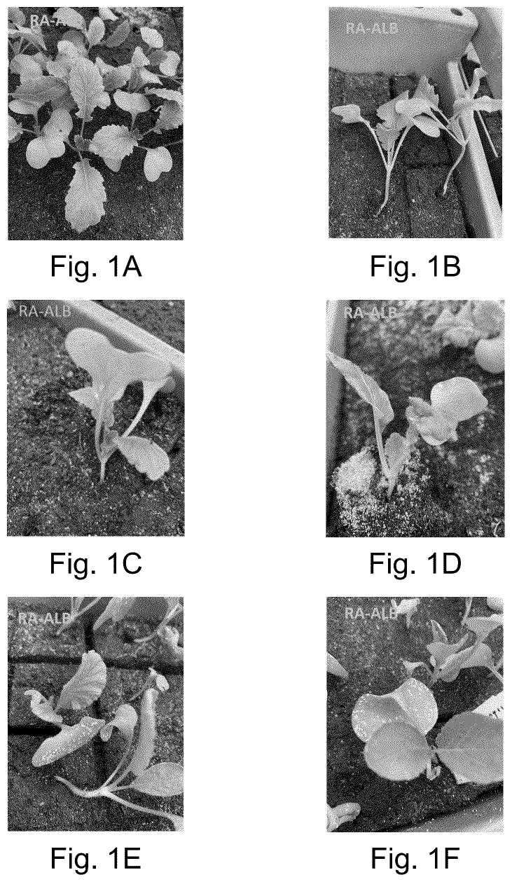

### DETAILED DESCRIPTION OF THE INVENTION

The gene of the invention is preferably a gene which encodes a protein that confers resistance to Albugo candida when expressed in a Raphanus sativus plant, wherein said gene comprises a nucleotide sequence comprising a coding sequence according to SEQ ID No. 2, or a nucleotide sequence which has in order of increased preference at least 70%, 75%, 80%, 83%, 85%, 86%, 87%, 88%, 89%, 90%, 91%, 92%, 93%, 94%, 95%, 96%, 97%, 98%, 99% sequence identity to SEQ ID No. 2, and wherein the coding sequence comprises a thymine on position 2240 of SEQ ID No. 2 or a thymine on a corresponding position of a homologous sequence that has at least 70%, 75%, 80%, 83%, 85%, 86%, 87%, 88%, 89%, 90%, 91%, 92%, 93%, 94%, 95%, 96%, 97%, 98%, 99% sequence identity to SEQ ID No. 2.

The gene of the invention, when present in a plant, in particular a Raphanus sativus, confers resistance to Albugo candida. The Albugo candida resistance conferred by the gene of the invention inherits in a dominant fashion.

As used herein, sequence identity is the percentage of nucleotides or amino acids that is identical between two sequences after proper alignment of those sequences. The person skilled in the art is aware of how to align sequences, for example by using a sequence alignment tool such as BLAST®, which can be used for both nucleotide sequences and protein sequences. To obtain the most significant result, the best possible alignment that gives the highest sequence identity score should be obtained. The percentage sequence identity is calculated through comparison over the length of the shortest sequence in the assessment. In the present case, a nucleotide sequence represents a gene that at least comprises a start codon and a stop codon or encodes an amino acid sequence which comprises a complete protein encoded by such a gene.

Sequence similarity for an amino acid sequence is calculated using EMBOSS stretcher 6.6.0 (www.ebi.ac.uk/Tools/psa/emboss_stretcher), using the EBLOSUM62 matrix with settings Gap open penalty: 12 and Gap extend penalty: 2.

The invention further relates to a plant, preferably a plant of the species Raphanus sativus plant, wherein the plant comprises the gene of the invention in its genome. A plant comprising the gene of the invention in its genome is referred to herein as a ‘plant of the invention’.

In a further embodiment, the plant of the invention is an agronomically elite plant, preferably an agronomically elite Raphanus sativus plant.

In the context of this invention, an agronomically elite plant is a plant having a genotype that, as a result of human intervention, comprises an accumulation of distinguishable and desirable agronomic traits which allow a producer to harvest a product of commercial significance, preferably the agronomically elite plant of the invention is a plant of an inbred line or a hybrid.

As used herein, a plant of an inbred line is a plant of a population of plants that is the result of three or more rounds of selfing, or backcrossing; or which plant is a double haploid. An inbred line may e.g. be a parent line used for the production of a commercial hybrid.

As used herein, a hybrid plant is a plant which is the result of a cross between two different plants having different genotypes. More in particular, a hybrid plant is the result of a cross between plants of two different inbred lines. Such a hybrid plant may e.g. be a plant of an F₁ hybrid variety.

In one embodiment, the plant of the invention is a member of the subspecies Raphanus sativus subsp. sativus.

In one embodiment, the plant of the invention is a member of the species Raphanus sativus, excluding subspecies Raphanus sativus subsp. niger.

Seed of Raphanus sativus L. comprising the gene of the invention was deposited with the NCIMB under accession number NCIMB 43737.

The invention thus relates to plants grown from seed deposited under NCIMB accession numbers NCIMB 43737.

As used herein, resistance or susceptibility against Albugo candida is determined in a young plant test. Plants to be tested are raised in a greenhouse with a daytime/nighttime temperature regime of 15° C., with 14 hours Son-T light. Nine days after sowing, the young plants are moved to a climate chamber with a daytime/nighttime temperature regime of 14° C./12° C., 14 hours TL lighting per day, and a relative humidity of 80%. Also, nine days after sowing the plants are inoculated using a suspension of Albugo candida spores (100,000 spores/ml). After spraying the inoculum, the plants are covered with plastic for one night. A final assessment of the inoculated plants is done 30 days post sowing by visual scoring for the Albugo candida symptoms, based on the scale described in Table 2.

As used herein, a plant exhibiting no symptoms after having been exposed to the above-described disease test, is considered to be resistant against the Albugo candida isolate for which it is tested. A plant exhibiting a single pustule or just a couple of pustules after having been exposed to the above-described disease test, is considered to be partially resistant against the Albugo candida isolate for which it is tested. A plant of which the lobs and leaves are covered with white pustules after having been exposed to the above-described disease test, is considered to be susceptible to the Albugo candida isolate for which it is tested (See Table 2).

Another aspect of the invention relates to a seed capable of growing into a plant of the invention wherein said plant comprises the gene of the invention. The invention also relates to use of said seed for the production of a plant of the invention, by growing said seed into a plant.

Yet another aspect of the invention relates to a swollen hypocotyl (radish) harvested from a Raphanus sativus plant of the invention wherein said swollen hypocotyl comprises the gene of the invention.

The invention also relates to propagation material suitable for producing a plant of the invention, wherein the propagation material is suitable for sexual reproduction, and is in particular selected from a microspore, a pollen, an ovary, an ovule, an embryo sac and an egg cell, or is suitable for vegetative reproduction, and is in particular selected from a cutting, a root, a stem a cell, and a protoplast, or is suitable for tissue culture of regenerable cells or protoplasts, and is in particular selected from a leaf, a pollen, an embryo, a cotyledon, a hypocotyl, a meristematic cell, a root, a root tip, an anther, a flower, a seed and a stem, wherein the propagation material comprises the gene of the invention.

The invention further relates to a cell of a plant of the invention. Such a cell may either be in isolated form, or a part of the complete plant or parts thereof and still forms a cell of the invention because such a cell comprises the gene of the invention. Each cell of a plant of the invention carries the gene of the invention. A cell of the invention may also be a regenerable cell that can regenerate into a new plant of the invention.

The invention further relates to plant tissue of a plant of the invention, which comprises the gene of the invention. The tissue can be undifferentiated tissue or already differentiated tissue. Undifferentiated tissue is for example a stem tip, an anther, a petal, or pollen, and can be used in micro propagation to obtain new plantlets that are grown into new plants of the invention. The tissue can also be grown from a cell of the invention.

The invention further relates to a method for the production of a plant comprising the gene of the invention, which plant is resistant to Albugo candida, by using tissue culture or by using vegetative propagation.

The invention moreover relates to progeny of a plant, a cell, a tissue, or a seed of the invention, which progeny comprises the gene of the invention. Such progeny can in itself be a plant, a cell, a tissue, or a seed. The progeny can in particular be progeny of a plant of the invention, representative seeds of which were deposited under NCIMB number 43737. As used herein, progeny comprises the first and all further descendants from a cross with a plant of the invention, wherein a cross comprises a cross with itself or a cross with another plant, and wherein a descendant that is determined to be progeny comprises the gene of the invention. Descendants can be obtained through selfing and/or further crossing of the deposit. Progeny also encompasses material that is obtained by vegetative propagation or another form of multiplication.

The invention further relates to the germplasm of plants of the invention. The germplasm is constituted by all inherited characteristics of an organism and according to the invention encompasses at least the resistance trait of the invention. The germplasm can be used in a breeding program for the development of plants that show resistance to Albugo candida. The use of germplasm that comprises the gene of the invention in breeding is also part of the present invention.

The invention also relates to the use of the gene of the invention for producing a plant that is resistant to Albugo candida. The plant is preferably a plant that belongs to the Brassicaceae plant family, in particular a Raphanus sativus L. plant.

The current invention also relates to the use of a plant of the invention as a crop, as a source of seed or as a source of propagation material.

The invention further provides a marker for identifying a Raphanus sativus plant comprising the gene of the invention. Herein referred to as marker of the invention. Preferably, the marker of the invention detects a Thymine (T) on position 2240 of SEQ ID No. 2, or said marker detects a polymorphism, in particular a Thymine (T), on a corresponding position of a homologous sequence that has in order of increased preference at least 70%, 75%, 80%, 83%, 85%, 86%, 87%, 88%, 89%, 90%, 91%, 92%, 93%, 94%, 95%, 96%, 97%, 98%, 99% sequence identity to SEQ ID No. 2. More preferably, the marker of the invention is a nucleic acid molecule comprising a nucleotide sequence according to SEQ ID No. 4, as shown in Table 1. Most preferably, the marker of the invention is a nucleic acid molecule comprising a nucleotide sequence according to SEQ ID No. 5 as shown in Table 1. The wild type alleles that have been investigated in the research that led to the present invention all have Cytosine (C) on position 2240 of SEQ ID No. 2. This SNP can be detected with a marker comprising a nucleotide sequence according to SEQ ID No. 6 as shown in Table 1.

The use of the marker of the invention for identification of gene of the invention is also part of this invention.

The present invention further relates to a method for identifying a plant resistant to Albugo candida, wherein the method comprises the following steps:
a) detecting in the genome of a plant the gene of the invention, and/or
b) detecting a unique polymorphism in the gene of the invention, and optionally
c) testing of the plant comprising the gene of the invention for exhibiting resistance to Albugo candida.

In one embodiment, the unique polymorphism that is detected is the Thymine (T) on position 2240 of SEQ ID No. 2, preferably this is done using the marker of the invention having SEQ ID No. 5.

The invention further relates to a method for seed production comprising growing a plant from a seed of the invention that comprises the gene of the invention homozygously, allowing the plant to produce seed and harvesting the seed. Production of the seed is suitably done by selfing or by crossing with another plant that is optionally also a plant of the invention. The plant grown from the seed produced as described herein is resistant to Albugo candida.

The invention also relates to a method for producing hybrid seed, comprising crossing a first parent plant with a second parent plant and harvesting the resultant hybrid seed, wherein the first parent plant and/or the second parent plant is a plant of the invention comprising the gene of the invention. Preferably, at least one of the parent plants comprises the gene of the invention homozygously.

The invention also relates to the hybrid seed produced by the method described herein and a hybrid plant grown from said hybrid seed.

Transgenic techniques used for transferring nucleotide sequences between plants that are sexually incompatible can also be used to produce a plant of the invention, by transferring the gene of the invention from one species to another. Techniques that can suitably be used comprise general plant transformation techniques known to the skilled person, such as the use of an Agrobacterium-mediated transformation method. A plant of the deposit or a descendant thereof is a suitable source of the modified gene.

Introduction of the gene of the invention can also be done through introgression from a plant comprising said gene, for example from a plant, representative seed of which was deposited as NCIMB 43737, or from progeny thereof, or from another plant that is resistant to Albugo candida and in which the gene of the invention was identified. Breeding methods such as crossing and selection, backcrossing, recombinant selection, or other breeding methods that result in the transfer of a genetic sequence from a resistant plant to a susceptible plant can be used. A resistant plant can be of the same species or of a different and/or wild species. Difficulties in crossing between species can be overcome through techniques known in the art such as embryo rescue, or cis-genesis can be applied. Progeny of a deposit can be sexual or vegetative descendants of that deposit, which can be selfed and/or crossed, and can be of an F1, F2, or further generation as long as the descendants of the deposit still comprise the modified gene the invention as present in seed of that deposit. A plant produced by such method is also a part of the invention.

The invention also relates to a method for the production of a plant exhibiting resistance against Albugo candida, comprising the steps of:
a) crossing a first parent plant comprising the gene of the invention with a second parent plant to obtain an F1 population;
b) optionally performing one or more rounds of selfing and/or crossing with a plant from the F1 population to obtain a further generation;
c) selecting from the further generation a plant that comprises the gene as a resistant plant.

The invention also relates to a method for the production of a plant which is resistant to Albugo candida, said method comprising:
a) crossing a first parent plant comprising the gene of the invention with a second parent plant not comprising the gene of the invention;
b) backcrossing the plant resulting from step a) with the second parent plant for at least three generations;
c) selecting from the third or higher backcross population a plant that comprises at least the gene of the invention of the first parent plant of step a) as the plant which is resistant to Albugo candida.

The invention additionally provides for a method of introducing another desired trait into a plant that is resistant to Albugo candida, comprising:
a) crossing a plant comprising the gene of the invention with a second plant that comprises the other desired trait to produce F1 progeny;
b) optionally selecting in the F1 for a plant that comprises the resistance and the other desired trait;
c) crossing the optionally selected F1 progeny with one of the parents for at least three generations, to produce backcross progeny;
d) selecting backcross progeny comprising the resistance and the other desired trait; and
e) optionally repeating steps c) and d) one or more times in succession to produce selected fourth or higher backcross progeny that comprises the resistance and the other desired trait.

Optionally, selfing steps are performed after any of the crossing or backcrossing steps in above-described methods. Selection of a plant comprising the Albugo candida resistance and the other desired trait can alternatively be done following any crossing or selfing step of the method. The other desired trait can be selected from, but is not limited to, the following group: resistance to bacterial, fungal or viral diseases, insect or pest resistance, improved germination, plant size, plant type, improved shelf-life, water stress and heat stress tolerance, and male sterility. The invention includes a plant produced by this method and a swollen hypocotyl obtained therefrom.

Although the present invention and its advantages have been described in detail, it should be understood that various changes, substitutions and alterations can be made herein without departing from the spirit and scope of the invention as defined in the appended claims.

### Sequence Information

TABLE 1
Sequences.

| ID# | Description | Sequence |
|---|---|---|
| SEQ ID No. 1 | Genomic | TCAATCGCCAATCCCTTCTTTTCCTTAAAATTATCTAGCTCC |
| | | TCTCTCTCGATCTCTATGGCTCTCTCATTAGCTTCTTCTTCT |
| | | TCCTCTTCTCGCACTTGGGTGTACGATGTTTTCCCGAGCTT |
| | | CAGTGGGGCAGACGTCCGTGTTACTTTCCTCAGCCACTTTC |
| | | GAAAGGAGCTTGACCGGAAACTGATCATTTCTTTCAAAGA |
| | | CAACGAGATAGAGAGAAGCCGGTCACTGGATCCAGAGCTT |
| | | AAACAAGCCATAAGAGATTCGAGGATCGCCGTGGTTATCT |
| | | TCTCTGAAAACTACGCATCTTCAAGCTGGTGTCTAAATGAG |
| | | TTGCTGGAGATCGTCAAGTGCAGGGATGAGTGTGGTCAAA |
| | | TGGTGATACCTGTTTTCTACCGTTTGGATCCTTCCCATGTC |
| | | AGGAAACAAACTGATGACTTTGGACAGATCTTTGAAAAAA |
| | | CATGCCACAACGAAACGGAGGAGGTGAAAATGCAATGGA |
| | | GGAGAGCTTTGACAAATGTAGCTAATATCACCGGATTCCA |
| | | TTCTGTAAACTGGTACGGTTTTATGAAACCTTCATTCTTGA |
| | | ATAAAGATTGTTACATGTGTATGAATTTATGTTTCTCATTG |
| | | CTAGATTGTGAAATTATTTTTCTTGTTGATAGGGGTAACGA |
| | | AGCAAAAATGATTGAAGAAATTGTCAGTGATGTTTTGGAG |
| | | AAACTACTTTTAACTCCATCGAAGGATTCTGAGAACTTTGT |
| | | GGGCATCGAAGGTCATATCGCAGATCTGAGTGTATTGCTG |
| | | GAGTTGGAATTAGAGGAAGTGAGGATGGTTGGTTTATGGG |
| | | GTGCTTCAGGGATTGGCAAGACTACAATTGCAAGAGTTCT |
| | | GTTCAACCGACTTTCTCGACACTTTCAAGGTAGCATTTTCA |
| | | TTGACAGGGCTTTCATATCTAAGAGTACTGAAGTTTTCAGC |
| | | AGAGCTAATCCGGGCGACTACAGCATGATGTTGCATTTGC |
| | | AATCGAGATTCCTATCGAAAATTCTAGGGAAAAATGACAT |
| | | AGAGATAAGTCAATTAGGCGAGTTAGCAGGGAGGCTGAA |
| | | GCACCATAAAGTTCTTATTTTCATTGATGATTTCAACGACC |
| | | AAGTGGTGCTAGATTCCTTGGTTGGTCAAACTCAATGGTTT |
| | | GGAAGTGGGAGCAGAATCGTTGTGGTTACAAATGATAAGC |
| | | ATCATTTACGATCCCATGGGATTGATCACATTTACGAGGTT |
| | | TGTCTCCCATCTCAAAAGCTAGCTGTTGAGATGTTTTGTCG |
| | | ATCTGCTTTCAACGAAAACTCTCCACCTGAAGGTTTTGAGG |
| | | TGCTTGTAGCTGAAATTACAAGACTTGCGGGTAGTCTTCCT |
| | | TTAGGTCTTAGTGTTTTGGGTTCATCTCTACGGGGGAGGGA |
| | | TAAAGAGTACTGGGTAGATTTGTTGCCAACGCTTCAGACT |
| | | GGTATAGATGGGAAAATCGAGAGAACATTGAAAGTCAGCT |
| | | ACGAAGGATTAAGTAGCAAAGGAGATAAAACGCTATTTCG |
| | | ACATATCGCATGCCTTTTCAATGGTGAAAAAGTCACATAC |
| | | CTAAAGTTGTTGCTCGCAGATAGTGACTTGAGTGTTAATGT |
| | | TGGGATGGAAAACCTAGCTGATAAGTCCCTCATTCATGTA |
| | | AGTCCGTGGGGTCATGTGGAGATGCACCGTTTGCTACAAG |
| | | AGATGGGTAGAAGTATTGTTCGTCTTGACGAGCCTGGGAC |
| | | ACGAGAATTTCTTATGGACTCACAAGATATATGTGATGTA |
| | | CTCAGTGATGGCACTGTAAGTTATCTCATATATTCGTGCTC |
| | | ATCTCATTCATTACGTACTCAGTATATAAGATTTAGAGCAT |
| | | GCCATTTTTATTAGCAGAACGAATAGTTAGCATCTGTTAAG |
| | | TTTTTTTTTTTGTTTATATTTCTTATTGATATTATATAATTTT |
| | | CAGGGGACTCAAAAGATATTAGGTATATCACTGGACACCG |
| | | ATGAGATTGATGAGTTGCATATACATGAGAACGCCTTCAC |
| | | AGGGATGCGTAATCTCCGTTTCCTAAAATTTTACTCGAGAT |
| | | ATGGAGTTGGCACAGTGAAATTGCACATACCCAAAAGCTT |
| | | CGACTATTTGCCGCTTAAACTGAAATCATTGTGTTGGCCGA |
| | | GATATCCAATGAGATGTCTTCCTTCTACATTTCGTCCTGAA |
| | | AACCTCACCGAGCTTAAAATGCAGGGTAGCAAGCTCGAGA |
| | | AGCTGTGGGAAGGGGTTGTGGTAAGTTTTGAGAAGAATTG |
| | | TTATGTTATTAGTAGTGAGAGTATAATCCTTGTTTTATATTT |
| | | CTGGATGACAATCATGTTCTAATCATGAGCCTCTGTTTTGT |
| | | TGAGTACAGTCGCTTCCATGTCTCAAGAAGATGGATTTGA |
| | | GTTTTTCTCACAACCTGATAGAAATGCCAGATCTTTCAAAG |
| | | GCCACCAATCTGGAGACACTTGATCTTGACGATTGCTATA |
| | | GTTTGGTGAAGCTTCCTTCCTCTATCCCACATCCCAACAAA |
| | | CTGATGAGATTGCACCTGATGAATTGTCAAAATCTGGAGA |
| | | TTATTCCAATTGGCATTAGCCTCAAATCTCTCGAAGAGCTA |
| | | TATCTCGATGGATGCTCACGGTTGAGGAATTTTCCTCAAAT |
| | | CTTAACCAACATCGAAACGATCAATATAGACGAAACACGC |
| | | ATTGAAGAAATCCCTTCAAATTTGCATCTGGAGGATGGGC |
| | | GTCTCAGGGAACTCAGTATGCAAAACGCAAAGAGTAAAA |
| | | AACCATGGGAAAGGGTGAAGGTATGTAGTTCCAAACTTTA |
| | | CGCTTTATCCAACCTTTTTGTTTCCTAAAGTTATTCAGCTAT |
| | | AGTTAGCATATTAGTTTTGGATCGAACACAATTGTCGTTGC |
| | | TAGGTCGATCGAATTTTGTATAGACGTGTTTATCACCACGG |
| | | TATTAGCGTTTTGCTTCAATAGTAAGGATAAATACATTTAA |
| | | TTATTCTCACACTTTGTAGTAGTTAATCATTGTGGCAAGAC |
| | | ATTAACTAGCGTTTTTGTAGTTAGATTTGATTTCTAGACAC |
| | | TAATAAATAACAATTTAGATCATATACTAATTTGCTACTGC |
| | | AAACCCCTAAGGGGCAAAAACCCAAGGTTTGGTAACTGAA |
| | | GATGATATTTGTATAATTTTTTTTTCAGCCGCTTATTCTACT |
| | | CACGGCGATCATGTCTCCACCTTTGAAGTATTTGTATCTAT |
| | | CGGATATGCCAACCTTGGTGGAGCTCCCTTCTTCATTTCAG |
| | | AATCTCCATCAACTGACGCATTTGGAAATTACGGACTGCA |
| | | TAAATCTGGAGACTCTTCCCACCGGAATCAACCTCCGTTCT |
| | | CTCGATGATCTCTATCTCAATGGATGCTCAAGGTTGAGTAC |
| | | TTTTCCTGATATCTCAACCAACATCAAAGTCCTCAGCCTAA |
| | | TCGCAACAGCCATTGAAGATGTTCCTGGGTCAGTTGATAA |
| | | ATTCTCTGTGCTTGAAGAGCTAAGGATGGAAAGATGCATA |
| | | AATCTGAAGATTCTTCCCTCTGGTATCAACCTCGCATCCCT |
| | | CAAATTCTTAGGTTTCAGTGGATGCCCACAGTTGAGTACTT |
| | | TTCCAGATATCTCTACCAACATTGAAGTGCTCTATCTAAAC |
| | | GAAACAGCCATTGAAGAGGTTCCTTGTTGGGTTGAGAAAT |
| | | TCACCAGGCTTGGAGTCTTGGAGATGAAGGGATGCAATAA |
| | | ATTAAAATTTGTAAGCTTAAACATTTCTAAACTCAAACGTC |
| | | TTGGGAGAGTTGAGTTATCAGACTGCTTAAACTTGGATCA |
| | | AGAAGCTCTGAAAACATATTCAGATTGTCTACTGAAGTTG |
| | | TCAAGTGAAGAAGTGCCTTCATATTTCATTCACCGTACTAC |
| | | GGTAACCTCCTCCTTCTCCTCTTTAACCGTCCCTCTACTTCC |
| | | CAGCTCTCTCTCGCAGCCATTCCTCCAATTCACGGCTTGCC |
| | | TTGTGCATACTTGGGACAATATGTCAGAAATTGATGCTATG |
| | | CCTAGTTTTACAAACAGATTTTGGAACAGCTCTGATTCCTA |
| | | TGTTCAGGAACAAGACTTCTACGGAACATGTACGAGTGAT |
| | | AGGCATATGTTAACATTGGTCTATTATATCTTTCTAAGCAG |
| | | AGATAGGGATATCCTAGCTCAAATGAACTACACGCATGTC |
| | | GATGTGCAGCTTGATATTAAGAATCGTAGTCCCTCTGAGTT |
| | | CAAAGAGTGGGGTATACGACTAGACTCAGGCCCGGCCCTG |
| | | TACTAAAACTGGTAAAGCAATAGCTTTAGGCGCCACAGAT |
| | | AACATATATTTACAGGGCGCCAAATGTACGTAGAGTCACT |
| | | TTGCCTTGGTGGTTTGGCTGCTGTTGTTTTGTAGCAAGGTG |
| | | ATGAGTTCGACACCTCCTCCCTTTAATATTTTTTTGTTTTTC |
| | | TTTTGGAAATTAGTAATAAAGATTTGGCCTCAACGATTTGG |
| | | TTTCCTTTTCTAAACAAATTTAATTATTTTATTACAAAATTA |
| | | CATAGATGATATTTTATTAATAAATTGTTTATCTAGGTAAA |
| | | TTTTAATTATTCTAAAATAAAGTATTTTTTATTGGTTCTCCA |
| | | CTTCTCCC |
| SEQ ID No. 2 | CDS | ATGGCTCTCTCATTAGCTTCTTCTTCTTCCTCTTCTCGCACT |
| | | TGGGTGTACGATGTTTTCCCGAGCTTCAGTGGGGCAGACG |
| | | TCCGTGTTACTTTCCTCAGCCACTTTCGAAAGGAGCTTGAC |
| | | CGGAAACTGATCATTTCTTTCAAAGACAACGAGATAGAGA |
| | | GAAGCCGGTCACTGGATCCAGAGCTTAAACAAGCCATAAG |
| | | AGATTCGAGGATCGCCGTGGTTATCTTCTCTGAAAACTACG |
| | | CATCTTCAAGCTGGTGTCTAAATGAGTTGCTGGAGATCGTC |
| | | AAGTGCAGGGATGAGTGTGGTCAAATGGTGATACCTGTTT |
| | | TCTACCGTTTGGATCCTTCCCATGTCAGGAAACAAACTGAT |
| | | GACTTTGGACAGATCTTTGAAAAAACATGCCACAACGAAA |
| | | CGGAGGAGGTGAAAATGCAATGGAGGAGAGCTTTGACAA |
| | | ATGTAGCTAATATCACCGGATTCCATTCTGTAAACTGGGGT |
| | | AACGAAGCAAAAATGATTGAAGAAATTGTCAGTGATGTTT |
| | | TGGAGAAACTACTTTTAACTCCATCGAAGGATTCTGAGAA |
| | | CTTTGTGGGCATCGAAGGTCATATCGCAGATCTGAGTGTAT |
| | | TGCTGGAGTTGGAATTAGAGGAAGTGAGGATGGTTGGTTT |
| | | ATGGGGTGCTTCAGGGATTGGCAAGACTACAATTGCAAGA |
| | | GTTCTGTTCAACCGACTTTCTCGACACTTTCAAGGTAGCAT |
| | | TTTCATTGACAGGGCTTTCATATCTAAGAGTACTGAAGTTT |
| | | TCAGCAGAGCTAATCCGGGCGACTACAGCATGATGTTGCA |
| | | TTTGCAATCGAGATTCCTATCGAAAATTCTAGGGAAAAAT |
| | | GACATAGAGATAAGTCAATTAGGCGAGTTAGCAGGGAGG |
| | | CTGAAGCACCATAAAGTTCTTATTTTCATTGATGATTTCAA |
| | | CGACCAAGTGGTGCTAGATTCCTTGGTTGGTCAAACTCAAT |
| | | GGTTTGGAAGTGGGAGCAGAATCGTTGTGGTTACAAATGA |
| | | TAAGCATCATTTACGATCCCATGGGATTGATCACATTTACG |
| | | AGGTTTGTCTCCCATCTCAAAAGCTAGCTGTTGAGATGTTT |
| | | TGTCGATCTGCTTTCAACGAAAACTCTCCACCTGAAGGTTT |
| | | TGAGGTGCTTGTAGCTGAAATTACAAGACTTGCGGGTAGT |
| | | CTTCCTTTAGGTCTTAGTGTTTTGGGTTCATCTCTACGGGG |
| | | GAGGGATAAAGAGTACTGGGTAGATTTGTTGCCAACGCTT |
| | | CAGACTGGTATAGATGGGAAAATCGAGAGAACATTGAAA |
| | | GTCAGCTACGAAGGATTAAGTAGCAAAGGAGATAAAACG |
| | | CTATTTCGACATATCGCATGCCTTTTCAATGGTGAAAAAGT |
| | | CACATACCTAAAGTTGTTGCTCGCAGATAGTGACTTGAGT |
| | | GTTAATGTTGGGATGGAAAACCTAGCTGATAAGTCCCTCA |
| | | TTCATGTAAGTCCGTGGGGTCATGTGGAGATGCACCGTTTG |
| | | CTACAAGAGATGGGTAGAAGTATTGTTCGTCTTGACGAGC |
| | | CTGGGACACGAGAATTTCTTATGGACTCACAAGATATATG |
| | | TGATGTACTCAGTGATGGCACTGGGACTCAAAAGATATTA |
| | | GGTATATCACTGGACACCGATGAGATTGATGAGTTGCATA |
| | | TACATGAGAACGCCTTCACAGGGATGCGTAATCTCCGTTTC |
| | | CTAAAATTTTACTCGAGATATGGAGTTGGCACAGTGAAAT |
| | | TGCACATACCCAAAAGCTTCGACTATTTGCCGCTTAAACTG |
| | | AAATCATTGTGTTGGCCGAGATATCCAATGAGATGTCTTCC |
| | | TTCTACATTTCGTCCTGAAAACCTCACCGAGCTTAAAATGC |
| | | AGGGTAGCAAGCTCGAGAAGCTGTGGGAAGGGGTTGTGTC |
| | | GCTTCCATGTCTCAAGAAGATGGATTTGAGTTTTTCTCACA |
| | | ACCTGATAGAAATGCCAGATCTTTCAAAGGCCACCAATCT |
| | | GGAGACACTTGATCTTGACGATTGCTATAGTTTGGTGAAG |
| | | CTTCCTTCCTCTATCCCACATCCCAACAAACTGATGAGATT |
| | | GCACCTGATGAATTGTCAAAATCTGGAGATTATTCCAATTG |
| | | GCATTAGCCTCAAATCTCTCGAAGAGCTATATCTCGATGG |
| | | ATGCTCACGGTTGAGGAATTTTCCTCAAATCTTAACCAACA |
| | | TCGAAACGATCAATATAGACGAAACACGCATTGAAGAAAT |
| | | CCCTTCAAATTTGCATCTGGAGGATGGGCGTCTCAGGGAA |
| | | CTCAGTATGCAAAACGCAAAGAGTAAAAAACCATGGGAA |
| | | AGGGTGAAGCCGCTTATTCTACTCACGGCGATCATGTCTCC |
| | | ACCTTTGAAGTATTTGTATCTATCGGATATGCCAACCTTGG |
| | | TGGAGCTCCCTTCTTCATTTCAGAATCTCCATCAACTGACG |
| | | CATTTGGAAATTACGGACTGCATAAATCTGGAGACTCTTCC |
| | | CACCGGAATCAACCTCCGTTCTCTCGATGATCTCTATCTCA |
| | | ATGGATGCTCAAGGTTGAGTACTTTTCCTGATATCTCAACC |
| | | AACATCAAAGTCCTCAGCCTAATCGCAACAGCCATTGAAG |
| | | ATGTTCCTGGGTCAGTTGATAAATTCTCTGTGCTTGAAGAG |
| | | CTAAGGATGGAAAGATGCATAAATCTGAAGATTCTTCCCT |
| | | CTGGTATCAACCTCGCATCCCTCAAATTCTTAGGTTTCAGT |
| | | GGATGCCCACAGTTGAGTACTTTTCCAGATATCTCTACCAA |
| | | CATTGAAGTGCTCTATCTAAACGAAACAGCCATTGAAGAG |
| | | GTTCCTTGTTGGGTTGAGAAATTCACCAGGCTTGGAGTCTT |
| | | GGAGATGAAGGGATGCAATAAATTAAAATTTGTAAGCTTA |
| | | AACATTTCTAAACTCAAACGTCTTGGGAGAGTTGAGTTATC |
| | | AGACTGCTTAAACTTGGATCAAGAAGCTCTGAAAACATAT |
| | | TCAGATTGTCTACTGAAGTTGTCAAGTGAAGAAGTGCCTTC |
| | | ATATTTCATTCACCGTACTACGGTAACCTCCTCCTTCTCCTC |
| | | TTTAACCGTCCCTCTACTTCCCAGCTCTCTCTCGCAGCCATT |
| | | CCTCCAATTCACGGCTTGCCTTGTGCATACTTGGGACAATA |
| | | TGTCAGAAATTGATGCTATGCCTAGTTTTACAAACAGATTT |
| | | TGGAACAGCTCTGATTCCTATGTTCAGGAACAAGACTTCTA |
| | | CGGAACATGTACGAGTGATAGGCATATGTTAACATTGGTC |
| | | TATTATATCTTTCTAAGCAGAGATAGGGATATCCTAGCTCA |
| | | AATGAACTACACGCATGTCGATGTGCAGCTTGATATTAAG |
| | | AATCGTAGTCCCTCTGAGTTCAAAGAGTGGGGTATACGAC |
| | | TAGACTCAGGCCCGGCCCTGTAC |
| SEQ ID No. 3 | Amino acid | MALSLASSSSSSRTWVYDVFPSFSGADVRVTFLSHFRKELDR |
| | sequence | KLIISFKDNEIERSRSLDPELKQAIRDSRIAVVIFSENYASSSWC |
| | | LNELLEIVKCRDECGQMVIPVFYRLDPSHVRKQTDDFGQIFEK |
| | | TCHNETEEVKMQWRRALTNVANITGFHSVNWGNEAKMIEEI |
| | | VSDVLEKLLLTPSKDSENFVGIEGHIADLSVLLELELEEVRMV |
| | | GLWGASGIGKTTIARVLFNRLSRHFQGSIFIDRAFISKSTEVFS |
| | | RANPGDYSMMLHLQSRFLSKILGKNDIEISQLGELAGRLKHH |
| | | KVLIFIDDFNDQVVLDSLVGQTQWFGSGSRIVVVTNDKHHLR |
| | | SHGIDHIYEVCLPSQKLAVEMFCRSAFNENSPPEGFEVLVAEI |
| | | TRLAGSLPLGLSVLGSSLRGRDKEYWVDLLPTLQTGIDGKIER |
| | | TLKVSYEGLSSKGDKTLFRHIACLENGEKVTYLKLLLADSDL |
| | | SVNVGMENLADKSLIHVSPWGHVEMHRLLQEMGRSIVRLDE |
| | | PGTREFLMDSQDICDVLSDGTGTQKILGISLDTDEIDELHIHEN |
| | | AFTGMRNLRFLKFYSRYGVGTVKLHIPKSFDYLPLKLKSLCW |
| | | PRYPMRCLPSTFRPENLTELKMQGSKLEKLWEGVVSLPCLKK |
| | | MDLSFSHNLIEMPDLSKATNLETLDLDDCYSLVKLPSSIPHPN |
| | | KLMRLHLMNCONLEIIPIGISLKSLEELYLDGCSRLRNFPQILT |
| | | NIETINIDETRIEEIPSNLHLEDGRLRELSMQNAKSKKPWERVK |
| | | PLILLTAIMSPPLKYLYLSDMPTLVELPSSFQNLHQLTHLEITD |
| | | CINLETLPTGINLRSLDDLYLNGCSRLSTFPDISTNIKVLSLIAT |
| | | AIEDVPGSVDKFSVLEELRMERCINLKILPSGINLASLKFLGFS |
| | | GCPQLSTFPDISTNIEVLYLNETAIEEVPCWVEKFTRLGVLEM |
| | | KGCNKLKFVSLNISKLKRLGRVELSDCLNLDQEALKTYSDCL |
| | | LKLSSEEVPSYFIHRTTVTSSFSSLTVPLLPSSLSQPFLQFTACL |
| | | VHTWDNMSEIDAMPSFTNRFWNSSDSYVQEQDFYGTCTSDR |
| | | HMLTLVYYIFLSRDRDILAQMNYTHVDVQLDIKNRSPSEFKE |
| | | WGIRLDSGPALY |
| SEQ ID No. 4 | | AATTTGCATCTGGAGGATGGG |
| SEQ ID No. 5 | marker | YATAGACGAAACACGCATTGAAGAAATCCYTTCAAATTTG |
| | | CATCTGGAGGATGGGCGTCTCAGGGAACTCASTATGCAAA |
| | | AY |
| SEQ ID No. 6 | marker | YATAGACGAAACACGCATTGAAGAAATCCYTTCAAATTTG |
| | | CATCCGGAGGATGGGCGTCTCAGGGAACTCASTATGCAAA |
| | | AY |

The present invention will be further illustrated in the following Examples which are given for illustration purposes only and are not intended to limit the invention in any way.

### EXAMPLES

### Example 1—Albugo candida Bioassay

In the process of identifying new sources of Albugo candida resistance, various sources of radish plants were screened in an Albugo candida bioassay. Next to the potential resistance sources two positive controls (line S 19.42074 and inbred line NIZ-AC2 of Nickerson/Hazera) and one negative control (variety Rudolf of Bejo) were included in the bioassay. The bioassay screened for three different isolates of Albugo candida separately. The first isolate was gathered in The Netherlands (Westland), the second isolate was gathered in Germany (Pfalz), and the third isolate was gathered in, Australia (Daylesford). Line S 19.42074 is resistant to isolate 1 and 3, while inbred line NIZ-AC2 is resistant to isolate 1 and 2. Young plants of each of the genotypes were inoculated with Albugo candida.

All plants were raised in a greenhouse with a daytime/nighttime temperature regime of 15° C., with 14 hours Son-T light. Nine days after sowing the young plants were moved to a climate chamber with a daytime/nighttime temperature regime of 14° C./12° C., 14 hours TL lighting per day, and a relative humidity of 80%. Also, nine days after sowing the plants were inoculated using a suspension of Albugo candida spores (100,000 spores/ml). After spraying the inoculum, the plants were covered with plastic for one night. A final assessment of the inoculated plants was done 30 days post sowing by visual scoring for the Albugo candida symptoms, based on the scale described in Table 2.

TABLE 2
Albugo Plant Disease Test

| Disease | | |
|---|---|---|
| Score | Albugo symptoms on plants | |
| 1 | No signs of infection of Albugo candida. | Resistant |
| | Plants are totally healthy and no pustules | |
| | are visible. | |
| 2 | Minor infection (single pustule or just a couple of | Partial |
| | pustules) of Albugo candida. | resistance |
| 3 | Strong infection, lobs and leaf covered with white | Susceptible |
| | pustules of Albugo candida. | |

The screen resulted in the identification of a source accession 970917 (internal denomination) that is resistant to all three isolates used in the bioassay. The positive controls plants (line S 19.42074 and inbred line NIZ-AC2, not comprising the gene of the invention) score, as expected, only resistant to two out of the three isolates, and the negative control (Variety Rudolf) is fully susceptible to all three isolates. The results are summarized in Table 3.

TABLE 3
Resistant scores against different Albugo candida isolates

| Line/variety | Isolate 1 (NL) | Isolate 2 (D) | Isolate 3(AU) |
|---|---|---|---|
| Line 970917 | R | R | R |
| line S 19.42074 | R | S | R |
| inbred line NIZ- | R | R | S |
| AC2 | | | |
| Variety Rudolf | S | S | S |

### Example 2—Identification of Candidate Gene

In order to determine the underlying genetics of the observed resistance in accession 970917, a QTL mapping on an F2 population of a cross between a plant of accession 970917 and a plant of fully susceptible line 1081.C341 (internally developed proprietary line) was performed. The F2 population that was used for the QTL study contained 180 individuals. Each F2 plant was selfed and 30 F3 seeds from each selfing were sown for phenotyping with the three isolates as described in Example 1. A genetic map was constructed for which 166 polymorphic markers were used having a maximum spacing of 29 cM. On chromosome 6 one major QTL was detected with size of about 17.1 cM and 10.5 Mbp. The QTL had an explained variance of 78-90%.

Further fine-mapping was done in order to reduce the size of the QTL. The first round of fine-mapping was performed on the F3 individuals. A second round of fine-mapping was performed on recombinants in the F4. The QTL region became significantly smaller, approx. 38.8 kbp. However, the introgression from accession 970917 deviated significantly from the reference genome which hampered further fine-mapping. Therefore, the genomes of the parents were de novo assembled to better compare the sequences between the susceptible parent and the resistant parent. High molecular weight DNA from the plants was sequenced using Oxford Nanopore Technologies, and assembled using Canu (Koren, S. et al. (2017): Canu: Scalable and accurate long-read assembly via adaptive x-mer weighting and repeat separation. Genome Res. 27, 722-736). The sequences were polished using Racon (Vaser, R., Sović, I., Nagarajan, N. & Šikić, M. (2017): Fast and accurate de novo genome assembly from long uncorrected reads. Genome Res. 27, 737-746), Medaka (nanoporetech.github.io/medaka/), and finally Pilon (Walker, B. J. et al. Pilon (2014): An Integrated Tool for Comprehensive Microbial Variant Detection and Genome Assembly Improvement. PLoS One 9, e112963) using Illumina short reads from the same plant. Sequence comparison, structural annotation and confirmation with expression data led to the identification of the gene of invention.

### Example 3—Introduction of the Gene of the Invention into a Susceptible Parent Line

A plant of accession 970917 was crossed with 1081.C341. From the F2 three plants homozygous for the gene of the invention were selected and inbred to produce F3 seed. This seed was deposited with the NCIMB under deposit accession number NCIMB 43737.

A seed of the same F3 population was crossed with a plant of a susceptible parent line. The introduction of the gene of the invention was done by backcrossing with the susceptible parent line. The gene of the invention was followed by using a marker based on nucleotide sequences SEQ ID No. 5 and 6. In SEQ ID No. 5 and No. 6, the SNP of interest is on position 45. Susceptible variants of the gene of the invention that were analyzed all had a cytosine on position 45, while the resistant form carries a thymine on position 45. This position in SEQ ID No. 5 and 6 corresponds to position 2240 of SEQ ID No. 2, the CDS of the gene of the invention.

After four backcross-generations the gene of the invention was introgressed in the susceptible parent. Plants of this backcrossed line were subjected to the bioassay as described in Example 1. All plants of the backcrossed line scored fully resistant to all three isolates. The parent line can be combined with any other Raphanus sativus parent line to create a hybrid variety resistant to Albugo candida.

The invention is further described by the following numbered paragraphs:

1. A gene encoding a protein which confers resistance to Albugo candida when expressed in a Raphanus sativus plant, wherein said gene comprises:
a) a nucleotide sequence encoding a protein having an amino acid sequence, which has in order of increased preference at least 70%, 75%, 80%, 83%, 85%, 86%, 87%, 88%, 89%, 90%, 91%, 92%, 93%, 94%, 95%, 96%, 97%, 98%, 99%, 100% sequence similarity to SEQ ID No. 3; or
b) a nucleotide sequence comprising a coding sequence which has in order of increased preference at least 70%, 75%, 80%, 83%, 85%, 86%, 87%, 88%, 89%, 90%, 91%, 92%, 93%, 94%, 95%, 96%, 97%, 98%, 99%, 100% sequence identity to SEQ ID No. 2; or
c) a nucleotide sequence encoding at least the amino acid positions 15 to 192 of SEQ ID No. 3, the amino acid positions 209 to 432 of SEQ ID No. 3, and the amino acid positions 540 to 1023 of SEQ ID No. 3; or
d) a nucleotide sequence encoding an LRR domain comprising the amino acid positions 540 to 1023 of SEQ ID No. 3 or encoding an LRR domain which has in order of increased preference at least 95%, 96%, 97%, 98%, 99%, 100% sequence similarity to the amino acid sequence of the amino acid positions 540 to 1023 of SEQ ID No. 3.

2. The gene of paragraph 1, wherein the gene comprises a thymine on position 2240 of SEQ ID No. 2 or a thymine on a corresponding position of a homologous sequence that has at least 70%, 75%, 80%, 83%, 85%, 86%, 87%, 88%, 89%, 90%, 91%, 92%, 93%, 94%, 95%, 96%, 97%, 98%, 99% sequence identity to SEQ ID No. 2.

3. A protein encoded by the gene of paragraph 1 or 2.

4. Use of the gene of paragraphs 1 or 2 for producing a plant that is resistant against Albugo candida.

5. A marker for the identification of the gene of paragraphs 1 or 2, wherein the marker detects a thymine on position 2240 of SEQ ID No. 2, or wherein the marker detects a thymine on a corresponding position of a homologous sequence that has at least 70% sequence identity to SEQ ID No. 2.

6. The marker of paragraph 5, wherein the marker is a nucleic acid molecule comprising SEQ ID No. 4.

7. The marker of paragraph 5, wherein the marker is a nucleic acid molecule comprising SEQ ID No. 5.

8. Use of the marker of any one of paragraphs 5 to 7 for identification of a plant comprising the gene of the invention.

9. Method for identifying a plant resistant to Albugo candida, wherein the method comprises the following steps:
a) detecting in the genome of a plant the gene of paragraphs 1 or 2, and/or
b) detecting a unique polymorphism in the gene of paragraphs 1 or 2, and optionally
c) testing of the plant comprising the gene of the invention for exhibiting resistance to Albugo candida.

10. The method of paragraph 9, wherein the detecting of the unique polymorphism is done with the marker of any one of paragraphs 5 to 7.

11. A plant, comprising the gene of paragraphs 1 or 2, wherein the plant is resistant to Albugo candida.

12. The plant of paragraph 11, wherein the plant is an agronomically elite plant, in particular a hybrid variety or an inbred line.

13. The plant of paragraph 11 or 12, wherein the plant belongs to the species Raphanus sativus.

14. A seed capable of growing into a plant of any one paragraphs 11-13.

15. Propagation material suitable for producing a plant of any one of paragraphs 11 to 13, wherein the propagation material is suitable for sexual reproduction, and is in particular selected from a microspore, pollen, ovary, ovule, embryo sac and egg cell, or is suitable for vegetative reproduction, and is in particular selected from a cutting, root, stem cell, and protoplast, or is suitable for tissue culture of regenerable cells or protoplasts, which regenerable cells or protoplasts are in particular selected from a leaf, pollen, embryo, cotyledon, hypocotyl, meristematic cell, root, root tip, anther, flower and stem, and wherein the propagation material comprises the gene of paragraph 1.

16. A method for selecting a plant resistant to Albugo candida, comprising identifying the presence of the gene of paragraph 1 or 2, optionally testing the plant for resistance against Albugo candida, and selecting a plant comprising said gene, wherein said plant exhibits resistance to Albugo candida.

17. A method for producing a Raphanus sativus plant exhibiting resistance against Albugo candida comprising the steps of:
a) crossing a first parent plant comprising the gene of paragraph 1 or 2 with a second parent plant to obtain an F1 population;
b) optionally performing one or more rounds of selfing and/or crossing with a plant from the F1 population to obtain a further generation population;
c) selecting from the further generation population a plant that comprises the gene of paragraph 1 or 2 as a resistant plant.

18. A method for producing hybrid seed resistant to Albugo candida, comprising the steps of crossing a first parent plant with a second parent plant, wherein one or both parent plants are homozygous for the gene of paragraph 1 or 2 and harvesting the hybrid seed.

19. The hybrid seed produced by the method of paragraph 18.

20. A plant grown from the hybrid seed of paragraph 19.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A gene encoding a protein which confers resistance to Albugo candida when expressed in a Raphanus sativus plant, wherein said gene comprises:
a) a nucleotide sequence encoding a protein having an amino acid sequence, which has in order of increased preference at least 70%, 75%, 80%, 83%, 85%, 86%, 87%, 88%, 89%, 90%, 91%, 92%, 93%, 94%, 95%, 96%, 97%, 98%, 99%, 100% sequence similarity to SEQ ID No. 3; or
b) a nucleotide sequence comprising a coding sequence which has in order of increased preference at least 70%, 75%, 80%, 83%, 85%, 86%, 87%, 88%, 89%, 90%, 91%, 92%, 93%, 94%, 95%, 96%, 97%, 98%, 99%, 100% sequence identity to SEQ ID No. 2; or
c) a nucleotide sequence encoding at least the amino acid positions 15 to 192 of SEQ ID No. 3, the amino acid positions 209 to 432 of SEQ ID No. 3, and the amino acid positions 540 to 1023 of SEQ ID No. 3; or
d) a nucleotide sequence encoding an LRR domain comprising the amino acid positions 540 to 1023 of SEQ ID No. 3 or encoding an LRR domain which has in order of increased preference at least 95%, 96%, 97%, 98%, 99%, 100% sequence similarity to the amino acid sequence of the amino acid positions 540 to 1023 of SEQ ID No. 3.

2. The gene of claim 1, wherein the gene comprises a thymine on position 2240 of SEQ ID No. 2 or a thymine on a corresponding position of a homologous sequence that has at least 70%, 75%, 80%, 83%, 85%, 86%, 87%, 88%, 89%, 90%, 91%, 92%, 93%, 94%, 95%, 96%, 97%, 98%, 99% sequence identity to SEQ ID No. 2.

3. A protein encoded by the gene of claim 1.

4. A method for producing a plant that is resistant against Albugo candida comprising using the gene of claim 1.

5. A marker for the identification of the gene of claim 1, wherein the marker detects a thymine on position 2240 of SEQ ID No. 2, or wherein the marker detects a thymine on a corresponding position of a homologous sequence that has at least 70% sequence identity to SEQ ID No. 2.

6. The marker of claim 5, wherein the marker is a nucleic acid molecule comprising SEQ ID No. 4.

7. The marker of claim 5, wherein the marker is a nucleic acid molecule comprising SEQ ID No. 5.

8. A method for identifying a plant comprising a gene encoding a protein which confers resistance to Albugo candida when expressed in a Raphanus sativus plant comprising using the marker of claim 5.

9. Method for identifying a plant resistant to Albugo candida, wherein the method comprises the following steps:
a) detecting in the genome of a plant the gene of claim 1, and/or
b) detecting a unique polymorphism in the gene of claim 1, and optionally
c) testing of the plant comprising the gene of the invention for exhibiting resistance to Albugo candida.

10. The method of claim 9, wherein the detecting of the unique polymorphism in step (b) is done with a marker that detects a thymine on position 2240 of SEQ ID No. 2, or wherein the marker detects a thymine on a corresponding position of a homologous sequence that has at least 70% sequence identity to SEQ ID No. 2.

11. A plant comprising the gene of claim 1, wherein the plant is resistant to Albugo candida.

12. The plant of claim 11, wherein the plant is an agronomically elite plant, in particular a hybrid variety or an inbred line.

13. The plant of claim 11, wherein the plant belongs to the species Raphanus sativus.

14. A seed capable of growing into the plant of claim 11.

15. Propagation material suitable for producing a plant that is resistant to Albugo candida, wherein the propagation material is suitable for sexual reproduction, and is in particular selected from a microspore, pollen, ovary, ovule, embryo sac and egg cell, or is suitable for vegetative reproduction, and is in particular selected from a cutting, root, stem cell, and protoplast, or is suitable for tissue culture of regenerable cells or protoplasts, which regenerable cells or protoplasts are in particular selected from a leaf, pollen, embryo, cotyledon, hypocotyl, meristematic cell, root, root tip, anther, flower and stem, and wherein the propagation material comprises the gene of claim 1.

16. A method for selecting a plant resistant to Albugo candida, comprising identifying the presence of the gene of claim 1, optionally testing the plant for resistance against Albugo candida, and selecting a plant comprising said gene, wherein said plant exhibits resistance to Albugo candida.

17. A method for producing a Raphanus sativus plant exhibiting resistance against Albugo candida comprising the steps of:
a) crossing a first parent plant comprising the gene of claim 1 with a second parent plant to obtain an F1 population;
b) optionally performing one or more rounds of selfing and/or crossing with a plant from the F1 population to obtain a further generation population;
c) selecting from the further generation population a plant that comprises the gene of claim 1 as a resistant plant.

18. A method for producing hybrid seed resistant to Albugo candida, comprising the steps of crossing a first parent plant with a second parent plant, wherein one or both parent plants are homozygous for the gene of claim 1 and harvesting the hybrid seed.

19. The hybrid seed produced by the method of claim 18.

20. A plant grown from the hybrid seed of claim 19.


SEQUENCE LISTING (6 sequences)

SEQ ID NO: 1 - DNA, 4519 bp, Raphanus sativus
  misc_feature 1..4519: note = Genomic sequence
  source 1..4519: mol_type = genomic DNA
tcaatcgccaatcccttcttttccttaaaattatctagctcctctctctcgatctctatggctctctcattagcttcttcttcttcctcttctcgcacttgggtgtacgatgttttcccgagcttcagtggggcagacgtccgtgttactttcctcagccactttcgaaaggagcttgaccggaaactgatcatttctttcaaagacaacgagatagagagaagccggtcactggatccagagcttaaacaagccataagagattcgaggatcgccgtggttatcttctctgaaaactacgcatcttcaagctggtgtctaaatgagttgctggagatcgtcaagtgcagggatgagtgtggtcaaatggtgatacctgttttctaccgtttggatccttcccatgtcaggaaacaaactgatgactttggacagatctttgaaaaaacatgccacaacgaaacggaggaggtgaaaatgcaatggaggagagctttgacaaatgtagctaatatcaccggattccattctgtaaactggtacggttttatgaaaccttcattcttgaataaagattgttacatgtgtatgaatttatgtttctcattgctagattgtgaaattatttttcttgttgataggggtaacgaagcaaaaatgattgaagaaattgtcagtgatgttttggagaaactacttttaactccatcgaaggattctgagaactttgtgggcatcgaaggtcatatcgcagatctgagtgtattgctggagttggaattagaggaagtgaggatggttggtttatggggtgcttcagggattggcaagactacaattgcaagagttctgttcaaccgactttctcgacactttcaaggtagcattttcattgacagggctttcatatctaagagtactgaagttttcagcagagctaatccgggcgactacagcatgatgttgcatttgcaatcgagattcctatcgaaaattctagggaaaaatgacatagagataagtcaattaggcgagttagcagggaggctgaagcaccataaagttcttattttcattgatgatttcaacgaccaagtggtgctagattccttggttggtcaaactcaatggtttggaagtgggagcagaatcgttgtggttacaaatgataagcatcatttacgatcccatgggattgatcacatttacgaggtttgtctcccatctcaaaagctagctgttgagatgttttgtcgatctgctttcaacgaaaactctccacctgaaggttttgaggtgcttgtagctgaaattacaagacttgcgggtagtcttcctttaggtcttagtgttttgggttcatctctacgggggagggataaagagtactgggtagatttgttgccaacgcttcagactggtatagatgggaaaatcgagagaacattgaaagtcagctacgaaggattaagtagcaaaggagataaaacgctatttcgacatatcgcatgccttttcaatggtgaaaaagtcacatacctaaagttgttgctcgcagatagtgacttgagtgttaatgttgggatggaaaacctagctgataagtccctcattcatgtaagtccgtggggtcatgtggagatgcaccgtttgctacaagagatgggtagaagtattgttcgtcttgacgagcctgggacacgagaatttcttatggactcacaagatatatgtgatgtactcagtgatggcactgtaagttatctcatatattcgtgctcatctcattcattacgtactcagtatataagatttagagcatgccatttttattagcagaacgaatagttagcatctgttaagtttttttttttgtttatatttcttattgatattatataattttcaggggactcaaaagatattaggtatatcactggacaccgatgagattgatgagttgcatatacatgagaacgccttcacagggatgcgtaatctccgtttcctaaaattttactcgagatatggagttggcacagtgaaattgcacatacccaaaagcttcgactatttgccgcttaaactgaaatcattgtgttggccgagatatccaatgagatgtcttccttctacatttcgtcctgaaaacctcaccgagcttaaaatgcagggtagcaagctcgagaagctgtgggaaggggttgtggtaagttttgagaagaattgttatgttattagtagtgagagtataatccttgttttatatttctggatgacaatcatgttctaatcatgagcctctgttttgttgagtacagtcgcttccatgtctcaagaagatggatttgagtttttctcacaacctgatagaaatgccagatctttcaaaggccaccaatctggagacacttgatcttgacgattgctatagtttggtgaagcttccttcctctatcccacatcccaacaaactgatgagattgcacctgatgaattgtcaaaatctggagattattccaattggcattagcctcaaatctctcgaagagctatatctcgatggatgctcacggttgaggaattttcctcaaatcttaaccaacatcgaaacgatcaatatagacgaaacacgcattgaagaaatcccttcaaatttgcatctggaggatgggcgtctcagggaactcagtatgcaaaacgcaaagagtaaaaaaccatgggaaagggtgaaggtatgtagttccaaactttacgctttatccaacctttttgtttcctaaagttattcagctatagttagcatattagttttggatcgaacacaattgtcgttgctaggtcgatcgaattttgtatagacgtgtttatcaccacggtattagcgttttgcttcaatagtaaggataaatacatttaattattctcacactttgtagtagttaatcattgtggcaagacattaactagcgtttttgtagttagatttgatttctagacactaataaataacaatttagatcatatactaatttgctactgcaaacccctaaggggcaaaaacccaaggtttggtaactgaagatgatatttgtataatttttttttcagccgcttattctactcacggcgatcatgtctccacctttgaagtatttgtatctatcggatatgccaaccttggtggagctcccttcttcatttcagaatctccatcaactgacgcatttggaaattacggactgcataaatctggagactcttcccaccggaatcaacctccgttctctcgatgatctctatctcaatggatgctcaaggttgagtacttttcctgatatctcaaccaacatcaaagtcctcagcctaatcgcaacagccattgaagatgttcctgggtcagttgataaattctctgtgcttgaagagctaaggatggaaagatgcataaatctgaagattcttccctctggtatcaacctcgcatccctcaaattcttaggtttcagtggatgcccacagttgagtacttttccagatatctctaccaacattgaagtgctctatctaaacgaaacagccattgaagaggttccttgttgggttgagaaattcaccaggcttggagtcttggagatgaagggatgcaataaattaaaatttgtaagcttaaacatttctaaactcaaacgtcttgggagagttgagttatcagactgcttaaacttggatcaagaagctctgaaaacatattcagattgtctactgaagttgtcaagtgaagaagtgccttcatatttcattcaccgtactacggtaacctcctccttctcctctttaaccgtccctctacttcccagctctctctcgcagccattcctccaattcacggcttgccttgtgcatacttgggacaatatgtcagaaattgatgctatgcctagttttacaaacagattttggaacagctctgattcctatgttcaggaacaagacttctacggaacatgtacgagtgataggcatatgttaacattggtctattatatctttctaagcagagatagggatatcctagctcaaatgaactacacgcatgtcgatgtgcagcttgatattaagaatcgtagtccctctgagttcaaagagtggggtatacgactagactcaggcccggccctgtactaaaactggtaaagcaatagctttaggcgccacagataacatatatttacagggcgccaaatgtacgtagagtcactttgccttggtggtttggctgctgttgttttgtagcaaggtgatgagttcgacacctcctccctttaatatttttttgtttttcttttggaaattagtaataaagatttggcctcaacgatttggtttccttttctaaacaaatttaattattttattacaaaattacatagatgatattttattaataaattgtttatctaggtaaattttaattattctaaaataaagtattttttattggttctccacttctccc

SEQ ID NO: 2 - DNA, 3384 bp, Raphanus sativus
  misc_feature 1..3384: note = CDS
  source 1..3384: mol_type = genomic DNA
atggctctctcattagcttcttcttcttcctcttctcgcacttgggtgtacgatgttttcccgagcttcagtggggcagacgtccgtgttactttcctcagccactttcgaaaggagcttgaccggaaactgatcatttctttcaaagacaacgagatagagagaagccggtcactggatccagagcttaaacaagccataagagattcgaggatcgccgtggttatcttctctgaaaactacgcatcttcaagctggtgtctaaatgagttgctggagatcgtcaagtgcagggatgagtgtggtcaaatggtgatacctgttttctaccgtttggatccttcccatgtcaggaaacaaactgatgactttggacagatctttgaaaaaacatgccacaacgaaacggaggaggtgaaaatgcaatggaggagagctttgacaaatgtagctaatatcaccggattccattctgtaaactggggtaacgaagcaaaaatgattgaagaaattgtcagtgatgttttggagaaactacttttaactccatcgaaggattctgagaactttgtgggcatcgaaggtcatatcgcagatctgagtgtattgctggagttggaattagaggaagtgaggatggttggtttatggggtgcttcagggattggcaagactacaattgcaagagttctgttcaaccgactttctcgacactttcaaggtagcattttcattgacagggctttcatatctaagagtactgaagttttcagcagagctaatccgggcgactacagcatgatgttgcatttgcaatcgagattcctatcgaaaattctagggaaaaatgacatagagataagtcaattaggcgagttagcagggaggctgaagcaccataaagttcttattttcattgatgatttcaacgaccaagtggtgctagattccttggttggtcaaactcaatggtttggaagtgggagcagaatcgttgtggttacaaatgataagcatcatttacgatcccatgggattgatcacatttacgaggtttgtctcccatctcaaaagctagctgttgagatgttttgtcgatctgctttcaacgaaaactctccacctgaaggttttgaggtgcttgtagctgaaattacaagacttgcgggtagtcttcctttaggtcttagtgttttgggttcatctctacgggggagggataaagagtactgggtagatttgttgccaacgcttcagactggtatagatgggaaaatcgagagaacattgaaagtcagctacgaaggattaagtagcaaaggagataaaacgctatttcgacatatcgcatgccttttcaatggtgaaaaagtcacatacctaaagttgttgctcgcagatagtgacttgagtgttaatgttgggatggaaaacctagctgataagtccctcattcatgtaagtccgtggggtcatgtggagatgcaccgtttgctacaagagatgggtagaagtattgttcgtcttgacgagcctgggacacgagaatttcttatggactcacaagatatatgtgatgtactcagtgatggcactgggactcaaaagatattaggtatatcactggacaccgatgagattgatgagttgcatatacatgagaacgccttcacagggatgcgtaatctccgtttcctaaaattttactcgagatatggagttggcacagtgaaattgcacatacccaaaagcttcgactatttgccgcttaaactgaaatcattgtgttggccgagatatccaatgagatgtcttccttctacatttcgtcctgaaaacctcaccgagcttaaaatgcagggtagcaagctcgagaagctgtgggaaggggttgtgtcgcttccatgtctcaagaagatggatttgagtttttctcacaacctgatagaaatgccagatctttcaaaggccaccaatctggagacacttgatcttgacgattgctatagtttggtgaagcttccttcctctatcccacatcccaacaaactgatgagattgcacctgatgaattgtcaaaatctggagattattccaattggcattagcctcaaatctctcgaagagctatatctcgatggatgctcacggttgaggaattttcctcaaatcttaaccaacatcgaaacgatcaatatagacgaaacacgcattgaagaaatcccttcaaatttgcatctggaggatgggcgtctcagggaactcagtatgcaaaacgcaaagagtaaaaaaccatgggaaagggtgaagccgcttattctactcacggcgatcatgtctccacctttgaagtatttgtatctatcggatatgccaaccttggtggagctcccttcttcatttcagaatctccatcaactgacgcatttggaaattacggactgcataaatctggagactcttcccaccggaatcaacctccgttctctcgatgatctctatctcaatggatgctcaaggttgagtacttttcctgatatctcaaccaacatcaaagtcctcagcctaatcgcaacagccattgaagatgttcctgggtcagttgataaattctctgtgcttgaagagctaaggatggaaagatgcataaatctgaagattcttccctctggtatcaacctcgcatccctcaaattcttaggtttcagtggatgcccacagttgagtacttttccagatatctctaccaacattgaagtgctctatctaaacgaaacagccattgaagaggttccttgttgggttgagaaattcaccaggcttggagtcttggagatgaagggatgcaataaattaaaatttgtaagcttaaacatttctaaactcaaacgtcttgggagagttgagttatcagactgcttaaacttggatcaagaagctctgaaaacatattcagattgtctactgaagttgtcaagtgaagaagtgccttcatatttcattcaccgtactacggtaacctcctccttctcctctttaaccgtccctctacttcccagctctctctcgcagccattcctccaattcacggcttgccttgtgcatacttgggacaatatgtcagaaattgatgctatgcctagttttacaaacagattttggaacagctctgattcctatgttcaggaacaagacttctacggaacatgtacgagtgataggcatatgttaacattggtctattatatctttctaagcagagatagggatatcctagctcaaatgaactacacgcatgtcgatgtgcagcttgatattaagaatcgtagtccctctgagttcaaagagtggggtatacgactagactcaggcccggccctgtac

SEQ ID NO: 3 - protein, 1128 aa, Raphanus sativus
  REGION 1..1128: note = Amino acid sequence
MALSLASSSSSSRTWVYDVFPSFSGADVRVTFLSHFRKELDRKLIISFKDNEIERSRSLDPELKQAIRDSRIAVVIFSENYASSSWCLNELLEIVKCRDECGQMVIPVFYRLDPSHVRKQTDDFGQIFEKTCHNETEEVKMQWRRALTNVANITGFHSVNWGNEAKMIEEIVSDVLEKLLLTPSKDSENFVGIEGHIADLSVLLELELEEVRMVGLWGASGIGKTTIARVLFNRLSRHFQGSIFIDRAFISKSTEVFSRANPGDYSMMLHLQSRFLSKILGKNDIEISQLGELAGRLKHHKVLIFIDDFNDQVVLDSLVGQTQWFGSGSRIVVVTNDKHHLRSHGIDHIYEVCLPSQKLAVEMFCRSAFNENSPPEGFEVLVAEITRLAGSLPLGLSVLGSSLRGRDKEYWVDLLPTLQTGIDGKIERTLKVSYEGLSSKGDKTLFRHIACLFNGEKVTYLKLLLADSDLSVNVGMENLADKSLIHVSPWGHVEMHRLLQEMGRSIVRLDEPGTREFLMDSQDICDVLSDGTGTQKILGISLDTDEIDELHIHENAFTGMRNLRFLKFYSRYGVGTVKLHIPKSFDYLPLKLKSLCWPRYPMRCLPSTFRPENLTELKMQGSKLEKLWEGVVSLPCLKKMDLSFSHNLIEMPDLSKATNLETLDLDDCYSLVKLPSSIPHPNKLMRLHLMNCQNLEIIPIGISLKSLEELYLDGCSRLRNFPQILTNIETINIDETRIEEIPSNLHLEDGRLRELSMQNAKSKKPWERVKPLILLTAIMSPPLKYLYLSDMPTLVELPSSFQNLHQLTHLEITDCINLETLPTGINLRSLDDLYLNGCSRLSTFPDISTNIKVLSLIATAIEDVPGSVDKFSVLEELRMERCINLKILPSGINLASLKFLGFSGCPQLSTFPDISTNIEVLYLNETAIEEVPCWVEKFTRLGVLEMKGCNKLKFVSLNISKLKRLGRVELSDCLNLDQEALKTYSDCLLKLSSEEVPSYFIHRTTVTSSFSSLTVPLLPSSLSQPFLQFTACLVHTWDNMSEIDAMPSFTNRFWNSSDSYVQEQDFYGTCTSDRHMLTLVYYIFLSRDRDILAQMNYTHVDVQLDIKNRSPSEFKEWGIRLDSGPALY

SEQ ID NO: 4 - DNA, 21 bp, Raphanus sativus
  misc_feature 1..21: note = Nucleic acid molecule marker
  source 1..21: mol_type = genomic DNA
aatttgcatctggaggatggg

SEQ ID NO: 5 - DNA, 82 bp, Raphanus sativus
  misc_feature 1..82: note = Nucleic acid molecule marker
  source 1..82: mol_type = genomic DNA
yatagacgaaacacgcattgaagaaatccyttcaaatttgcatctggaggatgggcgtctcagggaactcastatgcaaaay

SEQ ID NO: 6 - DNA, 82 bp, Raphanus sativus
  misc_feature 1..82: note = Nucleic acid molecule marker
  source 1..82: mol_type = genomic DNA
yatagacgaaacacgcattgaagaaatccyttcaaatttgcatccggaggatgggcgtctcagggaactcastatgcaaaay